SEQUENCE LISTING (6 sequences)

SEQ ID NO: 1 - protein, 316 aa, Plasmodium falciparum
MAPKAKIVLVGSGMIGGVMATLIVQKNLGDVVLFDIVKNMPHGKALDTSHTNVMAYSNCKVSGSNTYDDLAGADVVIVTAGFTKAPGKSDKEWNRDDLLPLNNKIMIEIGGHIKKNCPNAFIIVVTNPVDVMVQLLHQHSGVPKNKIIGLGGVLDTSRLKYYISQKLNVCPRDVNAHIVGAHGNKMVLLKRYITVGGIPLQEFINNKLISDAELEAIFDRTVNTALEIVNLHASPYVAPAAAIIEMAESYLKDLKKVLICSTLLEGQYGHSDIFGGTPVVLGANGVEQVIELQLNSEEKAKFDEAIAETKRMKALA

SEQ ID NO: 2 - protein, 316 aa, Plasmodium vivax
MTPKPKIVLVGSGMIGGVMATLIVQKNLGDVVMFDVVKNMPQGKALDTSHSNVMAYSNCKVTGSNSYDDLKGADVVIVTAGFTKAPGKSDKEWNRDDLLPLNNKIMIEIGGHIKNLCPNAFIIVVTNPVDVMVQLLFEHSGVPKNKIIGLGGVLDTSRLKYYISQKLNVCPRDVNALIVGAHGNKMVLLKRYITVGGIPLQEFINNKKITDEEVEGIFDRTVNTALEIVNLLASPYVAPAAAIIEMAESYLKDIKKVLVCSTLLEGQYGHSNIFGGTPLVIGGTGVEQVIELQLNAEEKTKFDEAVAETKRMKALI

SEQ ID NO: 3 - protein, 19 aa, Artificial sequence
  Synthetic Polypeptide
GFTKAPGKSDKEWNRDDLL

SEQ ID NO: 4 - protein, 18 aa, Artificial sequence
  Synthetic Polypeptide
NNKLISDAELEAIFDRTV

SEQ ID NO: 5 - protein, 18 aa, Artificial sequence
  Synthetic Polypeptide
NNKKITDEEVEGIFDRTV

SEQ ID NO: 6 - protein, 22 aa, Artificial sequence
  Synthetic Polypeptide
ASEFYNSENKTYDLDFKTPNND

(19) United States
(12) Patent Application Publication
(10) Pub. No.: US 2015/0285796 A1
(43) Pub. Date: Oct. 8, 2015

(54) Peptide and Antibody Test Material for Detecting Both Vivax Malaria and Falciparum Malaria

(71) Applicant: NATIONAL CENTER FOR GLOBAL HEALTH AND MEDICINE, Tokyo (JP)

(72) Inventors: Hiroyuki Oku, Kiryu-Shi (JP); Shinya Kitamura, Kiryu-Shi (JP); Keiichi Yamada, Kiryu-Shi (JP); Shigeyuki Kano, Tokyo (JP); Kazuhiko Yano, Tokyo (JP)

(21) Appl. No.: 14/439,693

(22) PCT Filed: Nov. 1, 2013
(86) PCT No.: PCT/JP2013/079666

§ 371 Date: Apr. 30, 2015

(30) Foreign Application Priority Data
Nov. 6, 2012 (JP) 2012-244637

(51) Int. Cl. G01N 33/569; C07K 16/20

(52) U.S. Cl. CPC G01N 33/56905; C07K 16/205; G01N 2469/10

(57) ABSTRACT

An object of the present invention is to provide a novel antigen peptide that can be used as an antibody test material for anti-malaria protozoa antibodies, which comprises as an active ingredient a peptide comprising the amino acid sequence of SEQ ID NO: 3.

### TECHNICAL FIELD

The present invention relates to a peptide for detecting malaria protozoa, particularly to a peptide antigen for determining antibody titers against both vivax malaria and falciparum malaria and an antibody test material comprising the same peptide. In particular, the present invention relates to a peptide and an antibody test material comprising the same peptide, which peptide can bind to antibodies against both vivax and falciparum malaria in blood samples from human and other animals.

### BACKGROUND ART

Malaria is estimated to account for 216 million infected individuals and 655 thousand deaths in the year 2010 according to the latest WHO World Malaria Report 2011 (Non-Patent Document 1). Ninety-one percent of the deaths were reported from Africa and, furthermore, 86% of the deaths occurred in children younger than 5 years old. As countermeasures against malaria had been developed on a global scale, the numbers of the infected individuals and the deceased individuals were significantly decreased by 17% and 26% relative to those in the year 2000, respectively. Not only developing countries but also countries with rapidly developing economies, such as India, China, Brazil, Thailand and the like, have wide malaria endemic areas. Therefore, malaria is one of the most prominent infectious diseases even in the current environment where countermeasures against an epidemic of malaria are progressing.

In Japan, malaria has been designated as an infectious disease in Category IV, to which the obligation to notify the number of all cases is applied. Indigenous malaria has been controlled since the year 1959. However, because movement of people has been increased as a result of Japan's economic development since then, cases of malaria in Japanese overseas travelers infected in endemic areas and cases of “imported malaria” in entrants who come from endemic areas and develop malaria in Japan have increased since the 1980s. Then, 154 malaria cases, which is the record for the largest number, were reported in the year 2000, whereas the annual case number is currently decreased and in a range of 50 to 60 due to the widespread knowledge about malaria prevention among overseas travelers, and the like. Moreover, in a neighboring country, South Korea, the once controlled “indigenous malaria” was revived, the case number of malaria infection was increased up to 4000 in the year 2000 and the annual case number fluctuates within a range of 1000 to 2000 even today. Imported malaria cases from South Korea to Japan have also reported (Non-Patent Document 2). Therefore, the issue of countermeasures against malaria is important not only in endemic areas but also at all the entry points of Japan.

Five species of protozoan parasites that belong to the genus Plasmodium and cause malaria in humans are falciparum malaria protozoa (Plasmodium falciparum), vivax malaria protozoa (Plasmodium vivax), malariae malaria protozoa (Plasmodium malariae), ovale malaria protozoa (Plasmodium ovale), and a part of primate malaria protozoa (Plasmodium knowlesi). Malaria protozoa are ingested into the body while a mosquito transmitting the protozoa is biting, and they enter the liver through the bloodstream (primary hepatic stage), proliferate by dividing in liver cells, and then are released into the bloodstream. The released parasites enter erythrocytes and repeat proliferation by dividing (erythrocytic cycle) and the parasites increased by proliferation are transmitted further by other mosquitos. Fever that is a symptom of malaria is induced through the erythrocytic cycle. In particular, when treatment is late, falciparum malaria poses a greater risk of severe illness and death compared to the other four species.

Moreover, malaria is not a simple health issue but also one of the causes of stagnated economic activities and social unrest in African countries. A correlation between a recent increase of individuals with malaria infection in endemic areas and tropical forest exploitation or the global warming is also pointed out. The number of individuals with malaria infection is expected to rise 50-80 million more per 2° C. of temperature rise through the global warming according to the reports of International Panel on Climate Change (1996 and 1998). Thus, re-emergence of malaria is feared even in the temperate regions including Japan, where malaria was eradicated by DDT spraying and hygiene measures after the Second World War.

Therefore, a reagent is desired to be developed, which can easily measure serum antibody titers to detect malaria infection or to confirm the effect of a vaccine against malaria.

By way of example of a method used worldwide, the titers of antibodies against malaria protozoa antigens in the serum/plasma of a malaria patient are determined by an IFAT and an ELISA. However, those methods are not easily performed in ordinary hospital laboratories because adjustment of antigens and operations are complicated. Commercial test kits based on the ELISA method are available but they are very expensive (ex. 50 dollar per one sample; DRG International Inc., USA) and therefore it is hard to say that the test kits are widely used. Thus, a technology is widely desired to be developed, which enables a measurement of antibody titers without requiring a precision measurement device and with a low cost (1 to 2 dollars per one sample) even at hospitals in malaria endemic areas or travel clinics in non-endemic areas.

A large number of kits to determine a past malaria history are required for control of malaria and demonstration of an epidemic of malaria to be ceasing, for example, in the Philippines (Palawan Island and the like). However, the IFAT method is used even today even though the measuring kit is very expensive. This method also needs one day for preparation of a slide glass for testing and fluorescence microscopy. In fact, a fluorescence microscope is very expensive (several million yen for one microscope), and only around 100 individuals can be observed in one round of the endemic area survey.

There has been an example of study aimed at creating a simple test kit but such a kit has not been achieved. This example is carried out by a slightly old method in which a reaction between latex (polystyrene) microparticles to which antigens derived from cultured falciparum malaria are physically adsorbed and the serum of a patient is observed under a microscope (Non-Patent Document 3). A similar study was attempted for a long time period (the 1980s to the 1990s) by Mamoru Suzuki and Kumiko Sato at Department of Parasitology, School of Medicine and Unit of Clinical Laboratory Science, School of Health Science, Gunma University. However, any practical use of the method has not been achieved because it is difficult for the method to distinguish a result from a reaction with a normal serum having no history of malaria infection. That is, this method is acceptable for investigational purpose only and not suitable for any practical use.

The inventors have previously reported a diagnosing material for malaria infection and a vaccine against malaria, which use an antigen derived from malaria protozoa (Patent Document 1), a method of producing an antigen peptide derived from malaria (Patent Document 2), and a method of producing microparticles in which malaria antigens are included (Patent Document 3).

However, a novel antigen has been further desired, which can determine a current state and recent history of malaria infection, that is, which is used in a simple test kit for the purpose of the endemic area survey and the like.

It is known that a peptide derived from a protein having a sequence well-conserved among the malaria protozoa species and thus containing a less number of antigen mutations is used as an antigen for malaria. Lactate dehydrogenase (LDH) peptides are known to be antigens for antibody production (Non-Patent Documents 4, 5).

### PRIOR ART DOCUMENTS

### Patent Documents

[Patent Document 1] Japanese Unexamined Patent Application Publication No. 2002-371098
[Patent Document 2] WO2006/035815
[Patent Document 3] Japanese Unexamined Patent Application Publication No. 2009-256324

### Non-Patent Documents

[Non-Patent Document 1] World Health Organization. Impact of malaria control. World Malaria Report 2011. Geneve: WHO Press, 2011: pp. 51-78.
[Non-Patent Document 2] Iwagami M, Itoda I, Hwang S Y, Kho W G, Kano S. Plasmodium vivax PCR genotyping of the first malaria case imported from South Korea into Japan. J Infect Chemother. 2009; vol. 15: pp. 27-33.
[Non-Patent Document 3] Anal. Chem. 2007, 79, 4690-4695
[Non-Patent Document 4] Peptides (2010) 31, 525-532
[Non-Patent Document 5] Immunobiology 2006; 211: 797-805

### SUMMARY OF THE INVENTION

An object of the present invention is to provide a novel antigen peptide that can be used as an antibody test material for anti-malaria protozoa antibodies.

The inventors studied intensively to resolve the above-described object and eventually found that antibody titers to malaria protozoa can be efficiently determined by using a peptide comprising the amino acid sequence of SEQ ID NO: 3 as an antigen peptide and finally completed the present invention.

That is, the present invention provides the following features.

<1> An antibody test material for anti-malaria protozoa antibodies, comprising as an active ingredient a peptide comprising the amino acid sequence of SEQ ID NO: 3.

<2> The antibody test material according to <1>, which is for testing anti-falciparum malaria antibodies and anti-vivax malaria antibodies.

<3> The antibody test material according to <1> or <2>, wherein the peptide is immobilized on a carrier.

<4> The antibody test material according to <3>, wherein the carrier is a polymer obtained by a polymerization reaction of compounds (I) and (II) below:

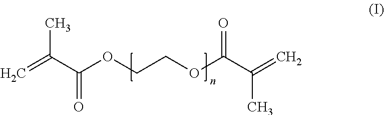

n represents an integer of 1 to 4,

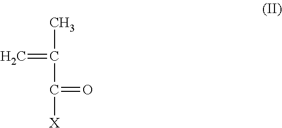

wherein X represents a halogen or —OY; Y represents an alkyl group, aromatic group, pyridyl group, quinolyl group, succinimide group, maleimide group, benzoxazole group, benzothiazole group, or benzotriazole group, wherein a hydrogen atom(s) in these groups may be substituted by a halogen(s).

<5> A test agent or diagnostic agent for infection with malaria protozoa, comprising the antibody test material according to any one of <1> to <4>.

<6> A test kit or diagnostic kit for infection with malaria protozoa, comprising the antibody test material according to any one of <1> to <4>.

<7> A method for testing or diagnosing infection with malaria protozoa, comprising the step of allowing the antibody test material according to any one of <1> to <4> to react with a sample derived from a subject infected by malaria protozoa.

Antibody titers to malaria protozoa can be efficiently determined by using the novel antigen peptide of the present invention.

The novel antigen peptide of the present invention is a type of antigen peptide that can determine a current state and recent history of falciparum malaria and vivax malaria, with which especially many patients are infected. The novel antigen peptide(s) of the present invention is/are less affected by a past (old) history of malaria infection and allow(s) to distinguish between a patient with fever caused not by malaria infection (or a patient suspected of malaria infection) and a patient infected with falciparum malaria or vivax malaria even in endemic areas. Therefore, the novel antigen peptide(s) of the present invention can be used in a simple test kit for the purpose of the endemic area survey and the like.

The novel antigen peptide(s) of the present invention can be immobilized on polymeric nanoparticles and thus antibody titers to malaria protozoa can be efficiently determined.

### BRIEF DESCRIPTION OF THE DRAWINGS

FIG. 1 shows schematic representations of the structures of falciparum malaria protozoa-derived LDH and vivax malaria protozoa-derived LDH. The sequences and the positions of artificial antigen peptides (a falciparum malaria-unique sequence part, a vivax malaria-unique sequence part, a common sequence part shared between falciparum malaria and vivax malaria) used in the Examples are indicated in the drawing.

FIG. 2 shows a HPLC chromatogram of the pLDH antigen (lot #2, crude product). Peaks at 7.9 min and 8.9 min in the drawing are attributed to an object of interest and a compound derived from the dehydrogenation of the object of interest, respectively.

FIG. 3 shows a HPLC chromatogram of the pLDH antigen (lot #2, the object of interest after solid-phase extracting purification using a SepPak column).

FIG. 4 shows an ESI-MS spectrum of the pLDH antigen (lot #2, the object of interest; the object of interest was isolated at 7.9 min by HPLC-based solid-phase extracting purification using a SepPak column). Calcd for [M+2H]²+, 1153.11. Found, 1153.9. Calcd for [M+3H]³⁺, 769.07. Found, 769.3. Calcd for [M+4H]⁴⁺, 577.06. Found: 577.5. Calcd for [M+5H]⁵⁺, 461.85. Found: 462.2.

FIG. 5 shows an ESI-MS spectrum of the pLDH antigen (lot #2, a by-product; the by-product, which was derived from the object of interest by detachment of two water molecules, was isolated at 8.9 min by HPLC-based solid-phase extracting purification using a SepPak column). Calcd for [M-H₂O+2H]²⁺, 1135.78. Found, 1136.0. Calcd for [M-H₂O+3H]³⁺, 757.57. Found, 757.6. Calcd for [M-H₂O+4H]⁴⁺, 568.39. Found: 568.6. Calcd for [M-H₂O+5H]⁵+, 454.92. Found: 454.9.

FIG. 6 shows a HPLC chromatogram of the pLDH antigen (lot #1, crude product). Peaks at 7.9 min and 8.9 min in the drawing are attributed to an object of interest and a compound derived from the dehydrogenation of the object of interest, respectively. Small peaks appearing at earlier elution times than 7.9 min and 8.9 min overlap each other. These peaks are attributed to deleted sequences having not more than 18 residues. Peaks appearing in a range of 13-14 min are attributed to products comprising a protecting group remaining on an Arg residue.

FIG. 7 shows results of measurements, in which the antibody titers in plasma samples derived from malaria patients and patients with fever collected in endemic areas of the Philippines was measured by using a partial peptide sequence of LDH, pLDH, as an antigen.

FIG. 8 shows results of measurements by the ELISA method, in which the antibody titers in plasma samples (at a serum dilution ratio of 64 times) derived from malaria patients and patients with fever collected in endemic areas of the Philippines were measured by using a partial peptide sequence of LDH, pLDH, as an antigen.

FIG. 9 shows results of measurements by the ELISA method, in which the antibody titers in plasma samples (at a serum dilution ratio of 256 times) derived from malaria patients and patients with fever collected in endemic areas of the Philippines were measured by using a partial peptide sequence of LDH, pLDH, as an antigen.

FIG. 10 shows results of measurements, in which the antibody titers in plasma samples derived from malaria patients and patients with fever collected in endemic areas of the Philippines were measured by using a partial peptide sequence unique in falciparum malaria-derived LDH, pfLDH, as an antigen.

FIG. 11 shows results of measurements, in which the antibody titers in plasma samples derived from malaria patients and patients with fever collected in endemic areas of the Philippines were measured by using a partial peptide sequence unique in vivax malaria-derived LDH, pvLDH, as an antigen.

FIG. 12 shows results of measurements, in which the antibody titers in serum samples derived from malaria patients and patients with fever collected in endemic areas of the Philippines were measured by using a partial peptide sequence of falciparum malaria-derived enolase, AD22, as an antigen.

FIG. 13 shows the molecular structure of the artificial antigen peptide (AD22)₄-MAP.

FIG. 14 shows the differences in sensitivity and false-positive rate in a case where 10 serum samples from patients with falciparum malaria and vivax malaria co-infection and 10 serum samples from patients with fever, which samples had been collected in endemic areas of the Philippines, were measured by a method of the present invention (pLDH microparticle) and a conventional method (AD22 microparticle) and subjected to an ROC analysis.

FIG. 15 shows the differences in sensitivity and false-positive rate in a case where 10 serum samples from patients with falciparum malaria infection and 10 serum samples from patients with fever, which samples had been collected in endemic areas of the Philippines, were measured by a method of the present invention (pLDH microparticle) and a conventional method (AD22 microparticle) and subjected to an ROC analysis.

FIG. 16 shows the differences in sensitivity and false-positive rate in a case where 10 serum samples from patients with falciparum malaria infection, 10 serum samples from patients with vivax malaria infection and 10 serum samples from patients with fever, which samples had been collected in endemic areas of the Philippines, were measured by a method of the present invention (pLDH microparticle) and a conventional method (AD22 microparticle) and subjected to an ROC analysis.

FIG. 17 shows results of measurements for the antibody titers in serum samples derived from malaria patients and patients with fever collected in endemic areas of the Philippines (the reactivity against (a) Pf antigen and (b) Pv antigen), wherein the measurements were performed by the existing diagnostic method, IFAT.

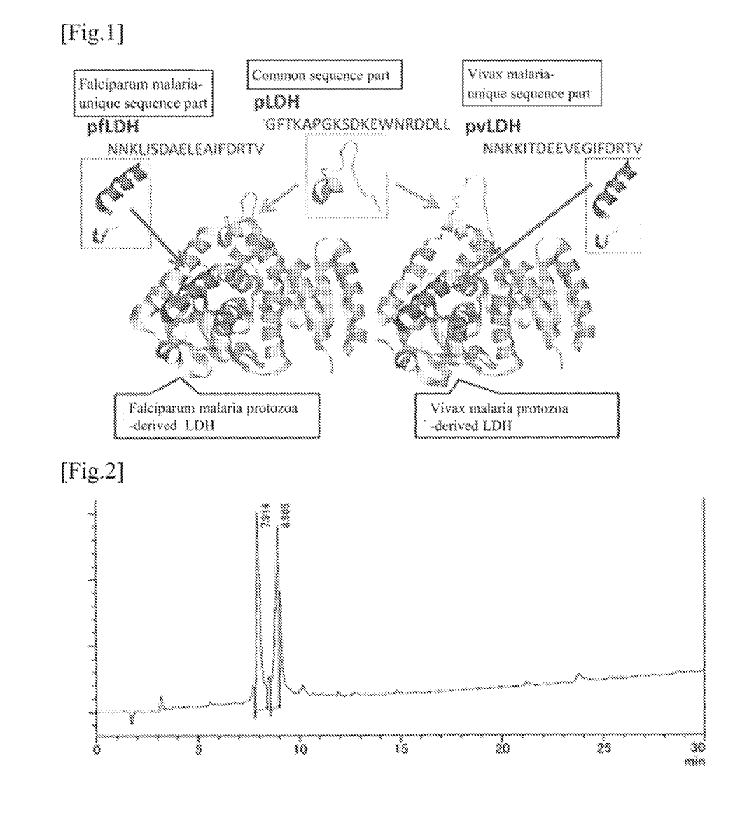

### DESCRIPTION OF THE EMBODIMENTS

Embodiments of the present invention are described below.

(1) Antibody Test Material for Anti-Malaria Protozoa Antibodies (Material for an Anti-Malaria Protozoa Antibody Test)

The present invention relates to an antibody test material for anti-malaria protozoa antibodies which comprises a peptide comprising the amino acid sequence of SEQ ID NO: 3 as an active ingredient, that is, as an antigen peptide.

The inventors focused on lactate dehydrogenase (LDH), which is a protein having a sequence well-conserved among the four human-infecting malaria protozoa species and thus containing a less number of antigen mutations, to design an antigen. Searched was a kind of antigen peptide that can determine a history of infection with falciparum malaria and vivax malaria, with which especially many patients are infected. Eventually obtained was a peptide comprising 19 residues and having a common sequence part shared between vivax malaria-derived LDH and falciparum malaria-derived LDH (pLDH; SEQ ID NO: 3).

Examples of the peptides comprising the amino acid sequence of SEQ ID NO: 3 include the peptide of SEQ ID NO: 3 as well as related peptides produced by substituting and/or deleting one or more of amino acids which constitute the peptide of SEQ ID NO: 3 and/or by inserting one or more amino acids into the peptide of SEQ ID NO: 3. The term “related peptide” refers to a peptide produced by substituting and/or deleting one or more of amino acids which constitute the peptide of SEQ ID NO: 3 and/or by inserting one or more amino acids into the peptide of SEQ ID NO: 3, and having the same activity in terms of the immune response as the peptide of the present invention. The number of amino acids subjected to substitution, deletion and/or insertion is not particularly limited but is preferably 1 to 3, and more preferably 1 to 2.

The antigen peptide comprising the amino acid sequence of SEQ ID NO: 3 is preferably a peptide comprising 19-21 residues.

The peptide may be labeled with a fluorescent material and the like, or may comprise one or more unnatural amino acids.

Since the sequence of the peptide of the present invention has been indicated in the present specification, the peptide can be prepared based on the sequence by any synthesis method including an organic chemical method, biochemical method and the like. As a method of preparing the peptide antigen of the present invention by an organic chemical method, the following examples can be used and are described in the Examples of the present specification: (1) a method to obtain the peptide antigen of the present invention by separately synthesizing several parts of the peptide antigen of the present invention and subsequently ligating them; (2) a method to obtain the peptide antigen of the present invention by allowing amino acids to be coupled sequentially on a solid-phase carrier and finally cleaving the resulting peptide. However, examples of a method of preparing the peptide antigen are not limited to these methods but the peptide antigen may be synthesized by using a synthesis procedure other than the methods disclosed in Examples of the present invention. Any of peptide synthesis methods available to those skilled in the art may be used, including, for example, the synthesis of the peptide compound of the present invention by an automated peptide synthesizer, and the like.

The peptide of the present invention can also be obtained through a biochemical method (i.e., recombinant DNA technology). For example, the peptide of the present invention is achieved by using an E. coli expression system to express LDH protein, which expression system uses a LDH protein-expressing vector comprising a DNA fragment coding for the whole sequence or a partial sequence of malaria protozoa-derived LDH gene, which is inserted into an E. coli expression vector downstream of the promoter of the vector. This expression vector can be constructed according to a known method (e.g., Sambrook and Russel, MOLECULAR CLONING: A LABORATORY MANUAL, 3rd edition (2001)). E. coli cells are transformed with this vector based on known methods and the protein is produced, and the produced protein can be collected and purified to obtain a peptide compound carrying a partial sequence of LDH.

Moreover, a peptide to be introduced into microparticles may be a form such that a plurality of sequences are connected each other in a linear form or a branched form. Examples of a carrier molecule which can be used to connect peptide sequences include natural proteins such as tetanus toxoid, ovalbumin, serum albumin, hemocyanin and the like. In cases where hemocyanin is used as a carrier, respective amino groups derived from the peptide and the carrier may be connected with glutaraldehyde, for example, by a method according to Boquet et al. (P. Boquet et al., Molecular Immunology (1988) vol. 19, pp. 1441-1549). For example, a synthetic polymeric carrier called MAP (multiple antigenic peptide) or lysine dendrimer may also be used.

Examples of the synthesis of a multimeric peptide using a synthetic polymeric carrier called MAP or lysine dendrimer include, for example, a method according to Tam (James P. Tam, Proc. Natl. Acad. Sci. USA. (1988) vol. 85, 5409-5413). Lysine molecules are allowed to react and bind to a dipeptide of β-alanine-cysteine (S-acetamidomethyl) immobilized on a resin in a stepwise manner by a known synthesis method and thereby a cross-linked body of interest can be prepared. That is, a conjugate comprising the dipeptide and one lysine molecule can be used as a branched peptide of a divalent form, a conjugate produced by a further reaction with lysine, which comprises three lysine resides, can be used as a branched peptide of a tetravalent from, and a conjugate produced by a still further reaction with lysine, which comprises seven lysine residues, can be used as an octavalent cross-linked body. Moreover, an octavalent body can also be obtained by oxidative deprotection of the acetamidomethyl group of the cysteine residue with iodine followed by formation of a disulfide bond. Constitutive amino acids of a peptide of interest are allowed to react and bind sequentially to these cross-linked bodies by an ordinary method and thereby a variety of multimeric peptides can be synthesized.

The peptide antigen of the present invention bound to anti-LDH antibodies can be detected by using a detection system known in the art, such as fluorescence ELISA method, agglutination assay and the like. This indicates that the peptide antigen of the present invention can be used as a novel peptide antigen which can be utilized as a material for the immunological diagnosis of falciparum malaria and vivax malaria. Thus, the peptide sequence of the present invention is useful as a diagnosing material for diagnosis of malaria, particularly as an artificial antigen which quite easily reacts with serum antibodies from a malaria patient.

The peptide antigen of the present invention can be provided as a material for immunological diagnosis by allowing the peptide antigen to be bound to, immobilized on, or adsorbed to the surface of a solid-phase material. Examples of the surface of the solid-phase material herein include, for example, but not limited to, the surface of a solid-phase material such as film, latex particle, polymeric microparticle, plastic plate or microbead. For example, the compound of the present invention bound to polymeric microparticles can be used for agglutination, as described below in details.

(2) Antigen-Immobilized Antibody Test Material

The present invention relates to the antibody test material in which the peptide(s) of the present invention is immobilized on a carrier obtained by a polymerization reaction of compounds (I) and (II) below:

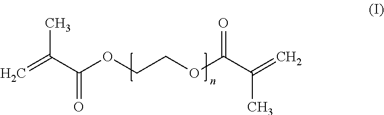

n represents an integer of 1 to 4.

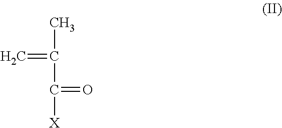

X represents a halogen (chlorine, fluorine, bromine, and the like) or —OY, wherein Y represents an alkyl group, aromatic group, pyridyl group, quinolyl group, succinimide group, maleimide group, benzoxazole group, benzothiazole group, or benzotriazole group and a hydrogen(s) in these groups may be substituted by a halogen(s) (chlorine, fluorine, bromine, and the like).

Examples of the alkyl group include, for example, groups such as methyl group, ethyl group, n-propyl group, isopropyl group, n-butyl group, t-butyl group, isobutyl group and sec-butyl group; examples of the aromatic group include, for example, groups such as phenyl group, 1-naphthyl group and 2-naphthyl group; examples of the pyridyl group include, for example, groups such as 2-pyridyl group, 3-pyridyl group and 4-pyridyl group; examples of the quinolyl group include, for example, groups such as 2-quinolyl group, 3-quinolyl group, 4-quinolyl group, 5-quinolyl group, 6-quinolyl group, 7-quinolyl group and 8-quinolyl group; examples of the benzoxazole group include, for example, 2-benzoxazole group and the like; examples of the benzothiazole group include, for example, 2-benzothiazole group and the like; examples of the benzotriazole group include, for example, 1-benzotriazole group and the like. Among those, phenyl group, 3-pyridyl group, 8-quinolyl group, succinimide group (OSu group), 2-benzothiazole group and 1-benzotriazole group (OBt group) are preferred because of higher activity of the resulting active esters.

A polymerization method can be performed by an ordinary radical polymerization method, and examples of such method include a method utilizing radiation (γ-rays) or a polymerization initiator.

A known radical polymerization initiator, such as azobisisobutyronitrile (AIBN), 1,1′-azobis(cyclohexanecarbonitrile) (ABCN) and the like, can be used as a polymerization initiator.

Any solvent is allowed as long as it dissolves each compound and the polymerization reaction proceeds in the solvent, and exemplary solvents include, for example, ethyl acetate, ethyl propionate, acetic acid, propionic acid, acetone, methyl ethyl isobutyl ketone (MIBK), dimethylformamide, dimethylacetamide, N-methylpyrrolidone, combinations of these solvents, and the like.

The diameter of a microparticle obtained by the polymerization reaction is preferably in a range of 0.1-10 μm. In addition, multiple kinds of compounds (I) and (II) may be used for the polymerization.

Introduction of the peptide to the surface of the microparticles can be performed by a reaction between an active ester group of the microparticle, —OY, and an amino group of the peptide. The amino group of the peptide may be a terminal amino group or a side-chain amino group. Moreover, a linker or carrier may be added to the peptide terminus and an amino group of the linker or carrier may be bound to the active ester group of the microparticle.

The ratio of the peptide introduced into the microparticle is preferably 0.05-2% by weight.

(3) Diagnostic and Test Agent and Kit

The present invention relates to a test or diagnostic agent for infection with malaria protozoa, which comprises the antibody test material of the present invention. Moreover, the present invention relates to a test or diagnostic kit for infection with malaria protozoa, which comprises the antibody test material of the present invention.

The peptide sequence of the present invention is an artificial antigen which quite easily reacts with serum antibodies from a patient with falciparum malaria infection and a patient with vivax malaria infection, and is useful as a test or diagnostic agent and a test or diagnostic kit for testing or diagnosing malaria infection.

The test or diagnostic agent and the test or diagnostic kit of the present invention can be composed of, in addition to the antibody test material of the present invention, a buffer and the like, and each element used in a test or diagnostic agent and a test or diagnostic kit known to those skilled in the art.

(4) Diagnostic and Test Method

Antibodies against malaria protozoa in a sample can be detected by using the antibody test material of the present invention.

The antibody test material of the present invention can detect anti-malaria protozoa-derived LDH antibodies, for example, in a blood sample of a subject and can be utilized as a test material for diagnosing malaria, investigating on a history of malaria infection, and confirming the maintenance of the antibody titers after vaccination.

As a sample, blood, serum, plasma and the like from a subject can be used.

A detection method is not particularly limited as long as the method detects a binding reaction, but examples of the detection method include the ELISA method, agglutination assay, a fluorescence-based detection method, a luminescence-based detection method, an ultraviolet-visible absorption-based detection method, an electrochemical detection method and the like. Among those, preferred is an agglutination assay using polymeric microparticles on which the peptide antigen of the present invention is immobilized.

The reaction of the peptide antigen with the antibody results in agglutination of the microparticles, which enables the antibodies to be detected by visual inspection.

Examples of the detectable antibodies include antibodies against the peptide sequence introduced into a microparticle (anti-peptide antibodies, such as anti-pLDH antibodies) or antibodies against a protein comprising this peptide sequence (anti-protein antibodies, such as anti-falciparum malaria-derived LDH antibodies and anti-vivax malaria-derived LDH antibodies, which identify the pLDH sequence), and, furthermore, antibodies against a peptide sequence or a protein which has one or more mutated amino acid residues and still retains a homology (antibodies raised against a relevant protein or peptide sequence of a closely related species).

Examples of the peptide sequence which has one or more mutated amino acid residues and still retains a homology include, for example, a sequence derived from relevant proteins of a closely related species, a sequence derived from the relevant proteins with several mutations, and an amino acid sequence derived from a relevant proteins with a high homology (with an amino acid identity of >60%).

### EXAMPLES

The present invention is described more specifically by way of examples but the present invention is not intended to be limited to these examples as long as the points of the present invention are followed.

### <Polymerization of a Polymer>

To perform a polymerization reaction, 0.4 g of the compound (i) below (commercial product, produced by Shin-Nakamura Chemical Co., Ltd.) and 0.1 g of the compound (ii) (a reaction product from methacrylic acid chloride and HOSu (N-hydroxysuccinimide) is used) in 10 mL of a solvent, ethyl propionate, were allowed to react at a room temperature of 25° C. for 3 hours under γ-irradiation (30 kGy):

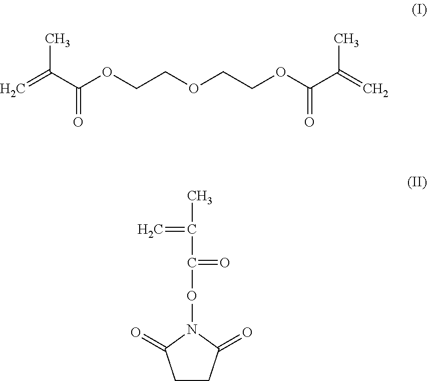 [SMILES: C=C(C)C(=O)OCCOCCOC(=O)C(=C)C ; C=C(C)C(=O)ON1C(=O)CCC1=O]

### <Immobilization of a Peptide>

As a peptide attached to the surface of a microparticle, the following peptide was used:

AD22 (having a molecular weight of 1.4 kD in a MAP form): Ala Ser Glu Phe Tyr Asn Ser Glu Asn Lys Thr Tyr Asp Leu Asp Phe Lys Thr Pro Asn Asn Asp (SEQ ID NO: 6).

The peptide (AD22)₄-MAP (MAP=multiple antigenic peptide; molecular weight: 1.4 kD; FIG. 13) was synthesized based on this antigen peptide sequence and using a manually operating synthesizer, and purified by a SepPack column (a disposable ODS column produced by Waters Co.).

### <Introduction of a Peptide>

Next, the antigen peptide of falciparum malaria protozoa (Comparative Example 1: (AD22)₄-MAP, 3 mg) was chemically bound to the surfaces of the microparticles with a total weight of 300 mg via an active ester group (succinimide group) to produce a test material.

The reaction was performed at 37° C. for 4 hours for the chemical binding and further at 37° C. for 20 hours for the physical adsorption.

### <Confirmation of the Introduction of the Peptide>

The progress of the reaction was confirmed by monitoring the amount of free HOSu (N-hydroxysuccinimide) with HPLC, which was generated along with the chemical binding of the above-described peptides.

### <Agglutination Test>

An agglutination test was performed using plasma samples from patients with falciparum malaria infection (Pf patients), plasma samples from normal volunteers (normal subjects) and plasma samples from patients (patients with fever) who were once suspected of being malaria patients because of fever and had blood sampling but later diagnosed to be negative for malaria from the result of an antigen-detection rapid diagnosis kit and the observation of smear specimens with a microscope, all of which samples were stored at the National Center for Global Health and Medicine.

In a 96-well plate, 50 μL each of one of the subject serum samples diluted with phosphate buffered saline (PBS) in a range of 16 to 2048 times was placed into 8 wells and 50 μL of a phosphate buffer control was placed into one well and finally 25 μL of the above-described microparticle (0.1 mg/mL) was added to each well. The 96-well plate was agitated for 1 min and subsequently allowed to stand for 8 hours at room temperature and thereby an agglutination reaction was detected.

Eventually, a positive agglutination image and a negative agglutination image were successfully obtained, which images showed a certain level of difference between the Pf patients and the normal subjects and between the Pf patients and the patients with fever.

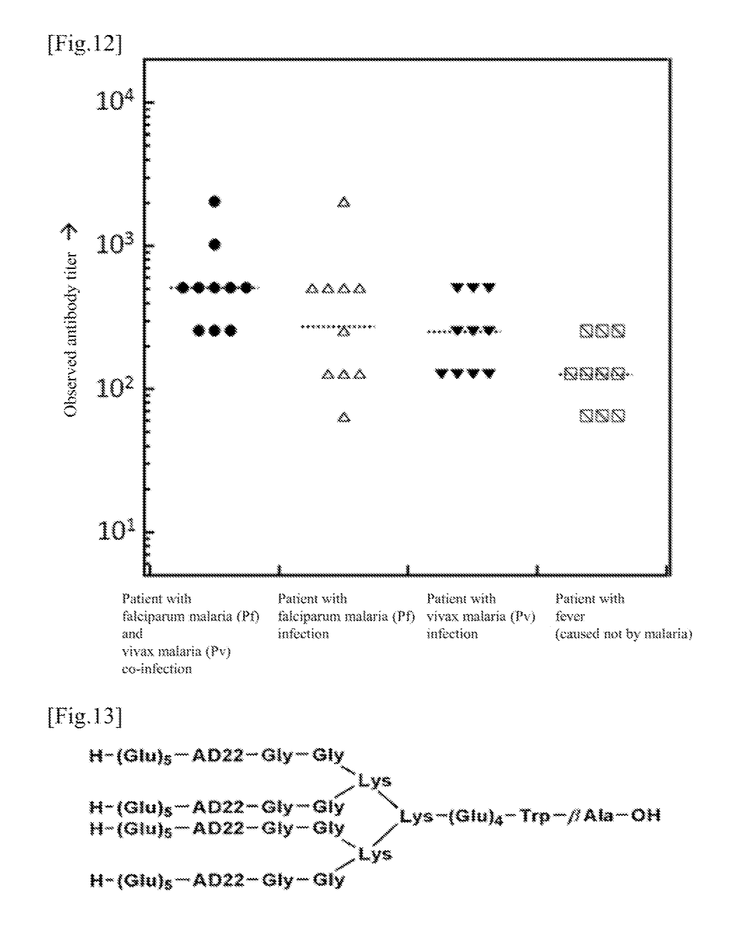

Furthermore, when the measurement was performed on serum samples from inhabitants in endemic areas of the Philippines (a total 40 samples of patient sera stored at Department of Parasitology, College of Public Health, University of the Philippines: 10 each from serum samples from patients with falciparum malaria and vivax malaria co-infection, serum samples from patients with falciparum malaria infection, plasma samples from patients with vivax malaria infection, and serum samples from patients (patients with fever) who were once suspected of being malaria patients because of fever and had blood sampling but later diagnosed to be negative for malaria from the result of an antigen-detection rapid diagnosis kit and the observation of smear specimens with a microscope), the measurement showed a vague difference in antibody titers between the malaria patients and the patients with fever (the patients not presently infected with malaria), which indicated a problem that the measurement fails to distinguish between a malaria patient and a non-malaria patient with sera from the endemic areas (FIG. 12). The reason for this problem is believed to be that the antibody titers to enolase reflects a past history of malaria infection for a relatively long time.

### Example 1

To obtain proteins derived from malaria protozoa for a peptide antigen sequence that enables a serum of a patient from an endemic area to be identified, the inventors focused on lactate dehydrogenase (LDH), which is a protein having a sequence well-conserved among the (four human-infecting) malaria species and thus containing a less number of antigen mutations, and designed an antigen. Searched was a kind of antigen peptides that can determine a history of infection with falciparum malaria and vivax malaria, with which especially many patients are infected.

Three kinds of antigen peptide sequences were selected from the amino acid sequences of malaria protozoa LDH species. That is, those are the falciparum malaria-unique sequence part peptide comprising 18 residues (pfLDH; SEQ ID NO: 4), the vivax malaria-unique sequence peptide comprising 18 residues (pvLDH; SEQ ID NO: 5), and the peptide comprising 19 residues and having a common sequence part shared between vivax malaria-derived LDH and falciparum malaria-derived LDH (pLDH; SEQ ID NO: 3).

<Synthesis of the pLDH Antigen>

Amino Acid Sequence:

(Gly-Phe-Thr-Lys-Ala-Pro-Gly-Lys-Ser-Asp-Lys-Glu-Trp-Asn-Arg-Asp-Asp-Leu-Leu) (SEQ ID NO: 3)-Lys

Composition formula: C₁₀₂H₁₆₁O₃₂N₂₉ (mw. 2304.19)

### Synthesis Procedure

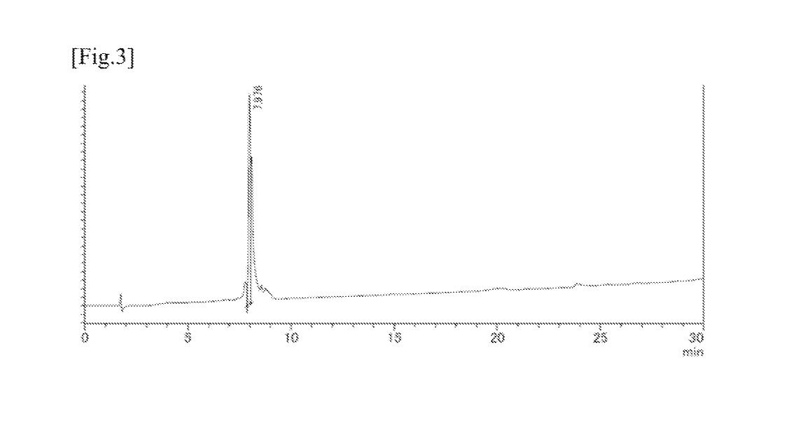

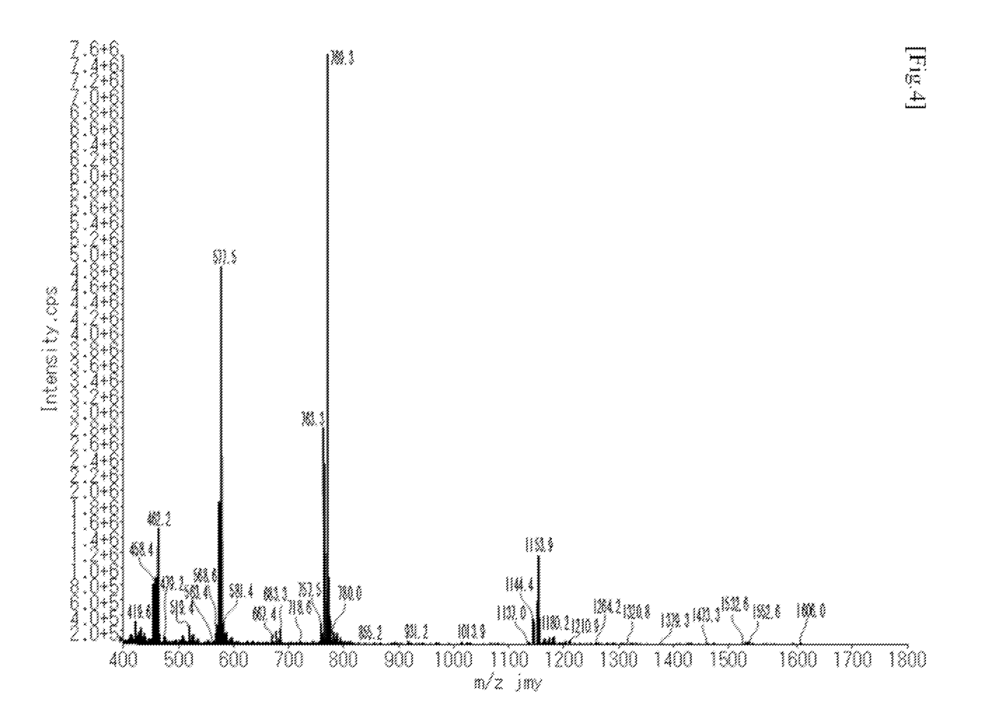

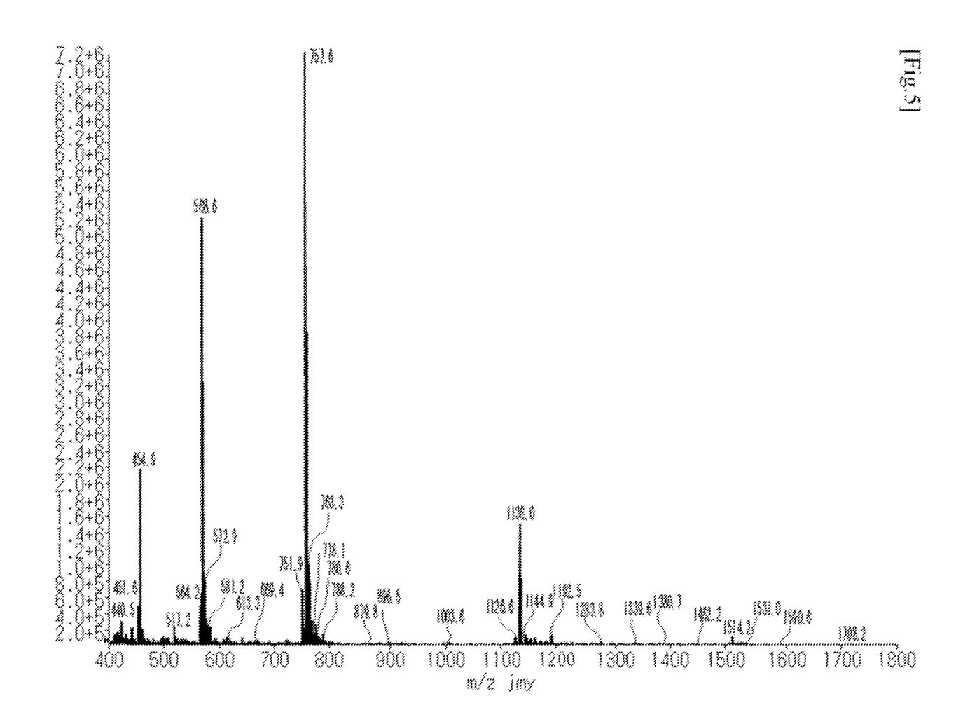

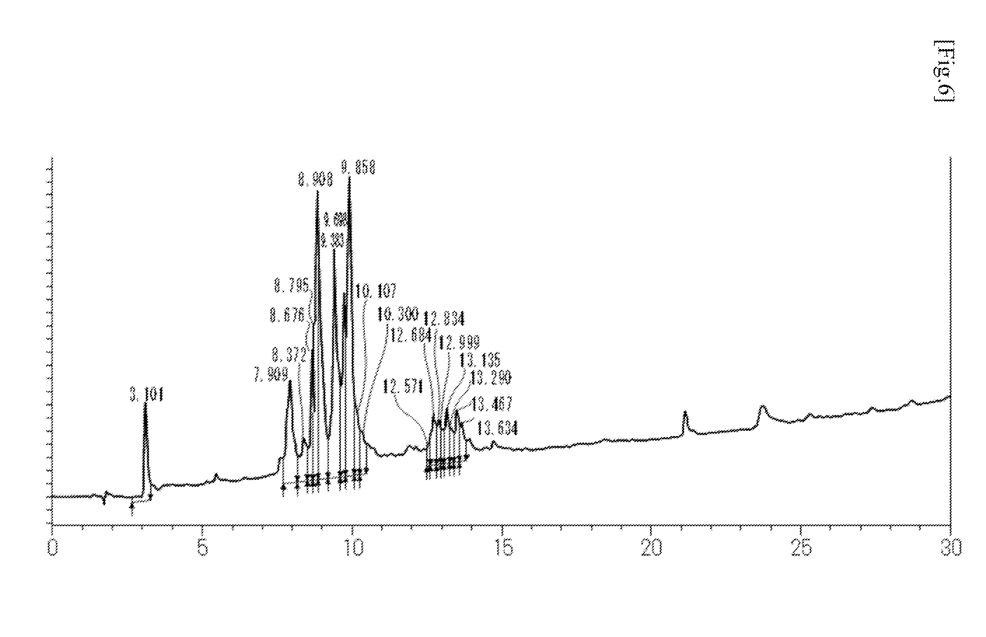

The synthesis of the pLDH antigen was performed using a Shimadzu PSSM8 automated peptide synthesizer by an Fmoc-based solid-phase synthesis method. As a resin used in the solid-phase synthesis, 54 mg of an Fmoc-Lys(Boc)-PEG-resin (0.18 mmol/g) was used. This resin was swelled in DMF at room temperature for 3 hours (Condition a) and then an Fmoc-deprotection reaction and a condensation reaction of protected amino acids were repeatedly performed. The amounts of Fmoc-protected amino acids used in the synthesis have been shown in Table 1 (0.10 mmol each, 10 equivalents). HCTU/HOBt/DIEA was used as a condensation reagent in an amount equimolar to the protected amino acids and a reaction time of 30 min was used in each condensation reaction. In the Fmoc deprotection, 3% DBU/DMF (Condition b) was used. After all amino acid condensation reactions, the resin was washed with DMF and CH₂Cl₂, mixed with 2 mL of a mixed reagent of trifluoroacetic acid, H₂O and triisopropylsilane in a ratio of 95:2.5:2.5, and allowed to stand for 20 hours at room temperature (Condition c) and thereby a resin cleavage reaction was performed. This solution was recovered and the resin was washed with CH₂Cl₂ and the solution was dried under reduced pressure to obtain a peptide. The obtained peptide was washed with cold diethyl ether and dried by vacuum drying to obtain a crude product (the yield of the crude product is listed in Table 2). An object of interest in this crude product was confirmed by HPLC (FIG. 2) and ESI-MS and the crude product was subsequently subjected to a solid-phase extraction using an ODS column (Waters SepPack ODS-5g) and eluted with 50 mL each of 15, 20, 25, and 30% of acetonitrile in water (with 0.1% trifluoroacetic acid) and collected in 10 mL fractions. Subsequently, each fraction was subjected to measurements by HPLC and ESI-MS and the object of interest was identified in the second fraction by elution with 20% acetonitrile in water and the fraction was lyophilized to give the object of interest (the yield of the purified product is listed in Table 2). The HPLC chromatogram and the ESI-MS spectrum of the purified product have been shown in FIGS. 3 and 4, respectively.

HPLC conditions

Analyzing device: Shimadzu LC-2010C-HT

Analyzing column: YMC-PACK ODS (4×100 mm, particle size: 3 μm)

Solvent conditions: 10%-70% acetonitrile in water (with 0.1% trifluoroacetic acid)

Analysis time: 30 min, Flow rate: 1 mL/min

ESI-MS conditions

Analyzing device: AP-SCIEX API-2000

Solvent: 10% acetonitrile in water (with 0.1% trifluoroacetic acid)

Flow rate: 10 mL/min

Examination of the Synthesis Conditions

As listed in Table 2, the yield of the object of interest was greatly increased from 12% to 50% by varying the three categories of Conditions a to c. Comparison of the HPLC chromatograms (FIGS. 2 and 6) indicates that the yield of the object of interest, which had no protected side chain remaining on the Arg residue, was greatly increased depending on the temperature and time in the cleavage reaction (Condition c). Furthermore, it was indicated that extending the swelling time (Condition a) allowed the resin for the solid-phase reaction to absorb the DMF solvent at a sufficient level and thereby the reaction reagent spread in the resin sufficiently, and changing the Fmoc-deprotecting reagent (Condition b) from piperidine to DBU increased the yield during the Fmoc-deprotection reaction and allowed no Fmoc group to remain on a peptide chain being synthesized and therefore resulted in reduced generation of deleted sequence species.

TABLE 1
Amounts of protected amino acid reagents
used in the synthesis of pLDH antigen

| number of condensed | | |
|---|---|---|
| amino acid | Fmoc-AA | Amount used |
| 1 | Gly | 30 mg |
| 2 | Phe | 40 mg |
| 3 | Thr(tBu) | 40 mg |
| 4 | Lys(Boc) | 47 mg |
| 5 | Ala | 31 mg |
| 6 | Pro | 34 mg |
| 7 | Gly | 30 mg |
| 8 | Lys(Boc) | 47 mg |
| 9 | Ser(tBu) | 39 mg |
| 10 | Asp(OtBu) | 42 mg |
| 11 | Lys(Boc) | 47 mg |
| 12 | Glu(OtBu) | 43 mg |
| 13 | Trp(Boc) | 53 mg |
| 14 | Asn(Trt) | 60 mg |
| 15 | Arg(Pbf) | 77 mg |
| 16 | Asp(OtBu) | 41 mg |
| 17 | Asp(OtBu) | 41 mg |
| 18 | Leu | 36 mg |
| 19 | Leu | 36 mg |

TABLE 2
Reaction conditions used in the synthesis of
pLDH antigen and comparison of yield between
a crude product and a purified product

| | Lot #1 | | Lot #2 and 3 | | Lot # 4 and 5 | |
|---|---|---|---|---|---|---|
| (Condition a) | 10 | min | 180 | min | 180 | min |
| Swelling time | | | | | | |
| (Condition b) | 30% | | 3% | DBU | 3% | DBU |
| Fmoc- | piperidine | | | | | |
| deprotecting | | | | | | |
| reagent | | | | | | |
| (Condition c) | 4° | C. | 20° | C. | 20° | C. |
| Cleavage | | | | | | |
| temperature | | | | | | |
| Theoretical yield | 23.2 | mg | 23.2 | mg | 23.2 | mg |
| Average yield of a | 19.3 | mg | 30.0 | mg | 22.9 | mg |
| crude product | | | | | | |

| | | | |
|---|---|---|---|
| Average yield of a | 2.9 mg | 11.5 mg | 11.5 mg |
| purified product | (12%) | (50%) | (50%) |
| (Yield) | | | |

### <Modification of the Antigen Peptide for the Polymeric Microparticle>

The chemical modification of the antigen peptides for the polymeric microparticle was performed according to the similar method of the above-described Comparative Example 1, except that the antigen peptide was not in a MAP form, and the antigen peptide and the polymeric microparticles were allowed to react at 37° C. in an incubator for 24 hours in each reaction. Then, the reaction was blocked by centrifugation. An aliquot of the reaction solution was sampled in the middle of the reaction time, at the time points of 4 and 24 hours, and the progress of the reaction was confirmed by detecting generated HOSu with reversed-phase HPLC.

### <Agglutination Test>

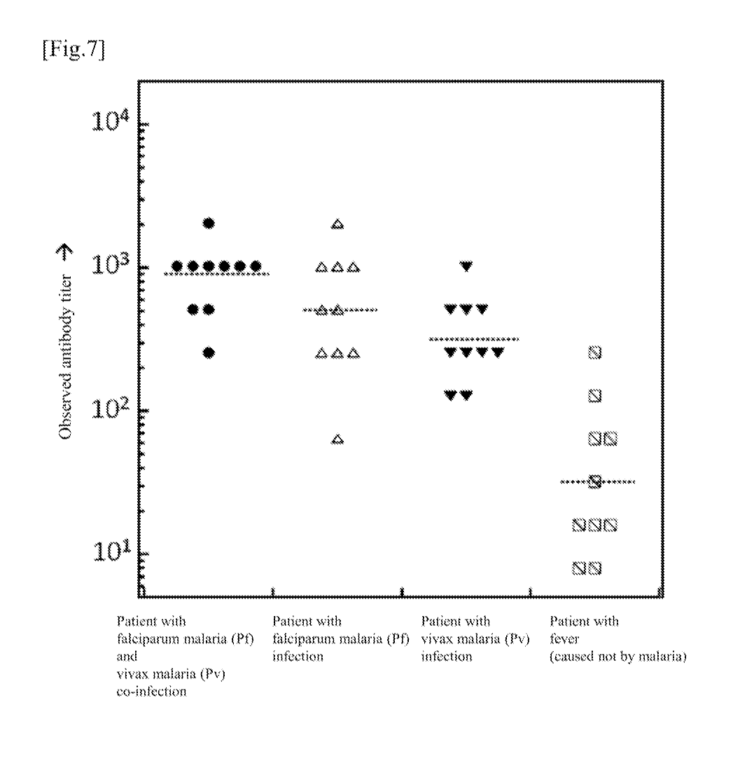

An agglutination test was performed using each of plasma samples from patients with falciparum malaria and vivax malaria co-infection plasma (Pf,Pv co-infection patients), patients with falciparum malaria infection (Pf patients), patients with vivax malaria infection (Pv patients), and patients who were once suspected of being malaria patients because of fever and had blood sampling (patients with fever), all of which samples were stored at College of Public Health, University of the Philippines Manila. In a 96-well plate, 25 μL each of one of the plasma samples diluted with phosphate buffered saline (PBS-0.1% tween 20) in a range of 16 to 32768 times was placed into 12 wells and finally 25 μL of the pLDH antigen-modified nanoparticle (0.1 mg/mL) was added to each well. The 96-well plate was agitated for 1 min and subsequently allowed to stand for 8 hours at room temperature and thereby an agglutination reaction was detected. Eventually, as shown in FIG. 7, antibody titers that indicated a significant difference between the malaria patients (Pf,Pv co-infection patients, Pf patients, Pv patients) and the patients with fever caused not by malaria were successfully measured.

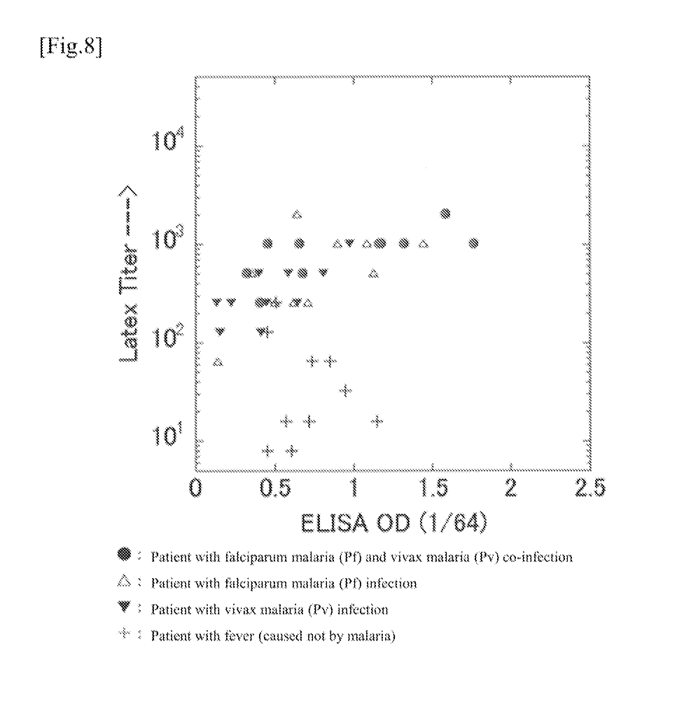

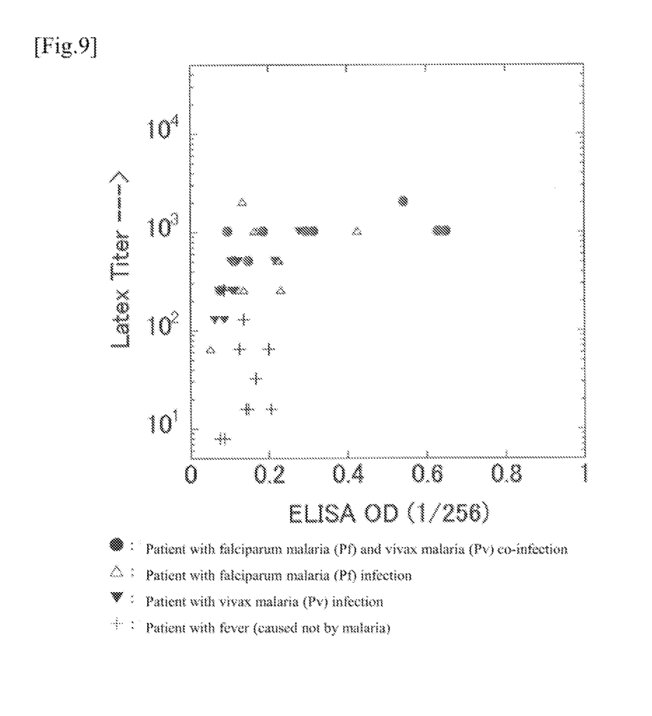

The obtained antibody titers were compared to those obtained by the conventional antibody titer test method, ELISA. In the ELISA method, diluted plasma samples as a primary antibody (in a dilution ratio of 1/64 and 1/256, 25 μL each), an HRP-modified anti-human IgG antibody as a secondary antibody (in a dilution ratio of 1/1000, 100 μL), and ABTS as a detection reagent (at a concentration of 0.7 mg/mL, 300 μL) were used with a 96-well microplate produced by NUNC (Immobilizer Amino plate), to which the antigen peptide had been linked (10 μg to each well). The absorbance at 405 nm in a microplate reader was used in the measurement of the antibody titers by the ELISA method, in which the plasma samples were diluted at a ratio of 1/64 and 1/256. As shown in FIG. 8 (in a dilution ratio of 64 times) and FIG. 9 (in a dilution ratio of 256 times), it was indicated that the measurement of the antibody titers using the nanoparticle correlated well to that using the conventional ELISA measurement method.

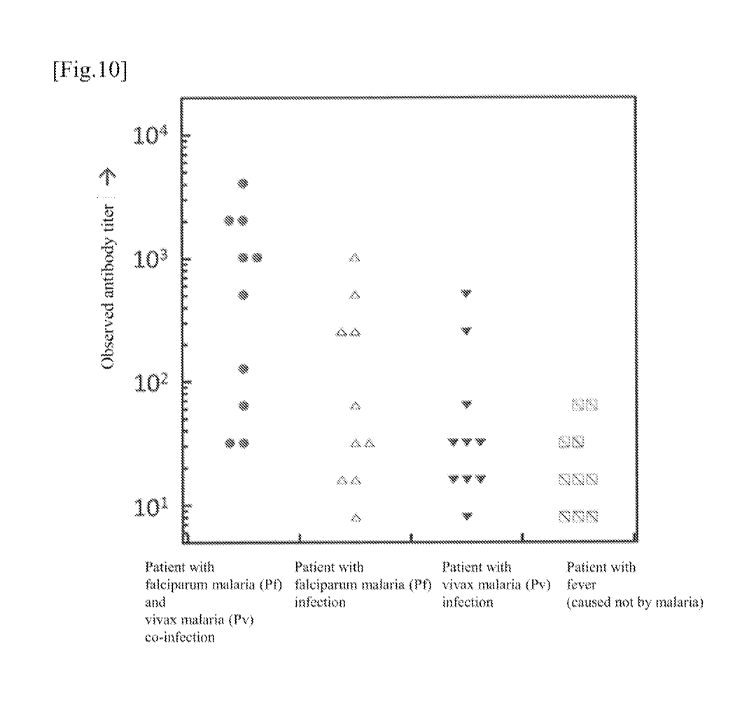

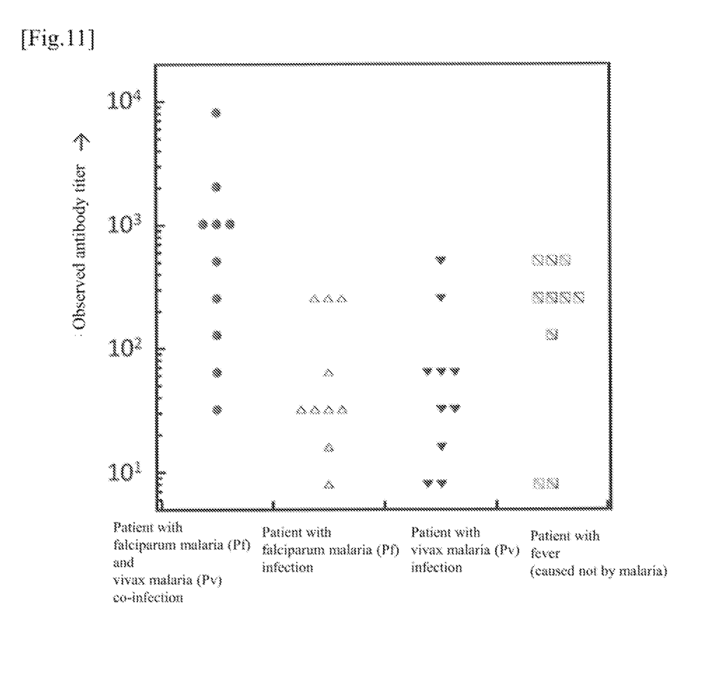

The measurement of the antibody titers in a similar method was performed using nanoparticles modified with the pfLDH antigen (FIG. 10) and the pvLDH antigen (FIG. 11), respectively. As shown in FIGS. 10 and 11, the method was not able to identify a significant difference between malaria patients (Pf,Pv co-infection patients, Pf patients, or Pv patients) and patients with fever caused not by malaria in these measurements. Accordingly, the nanoparticle modified with the pLDH antigen was indicated to be suitable for the diagnosis of malaria infection.

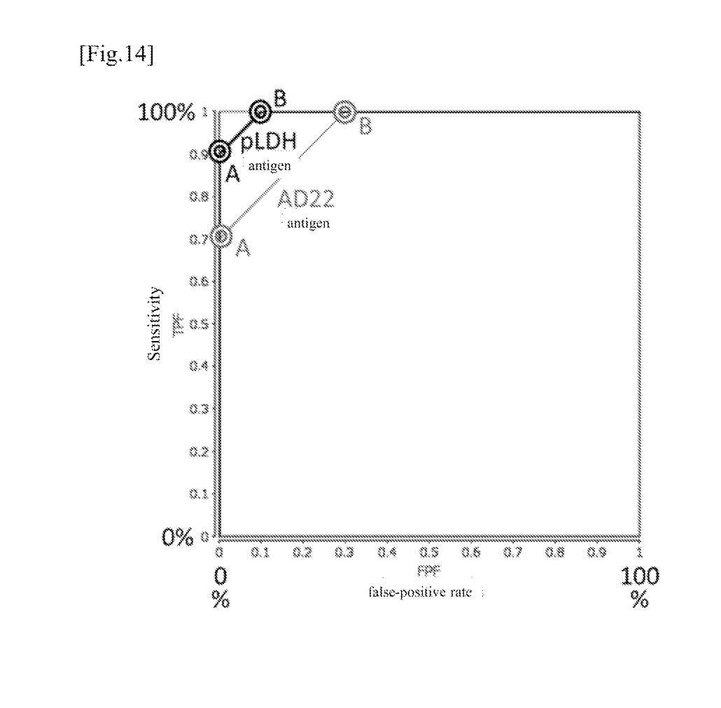

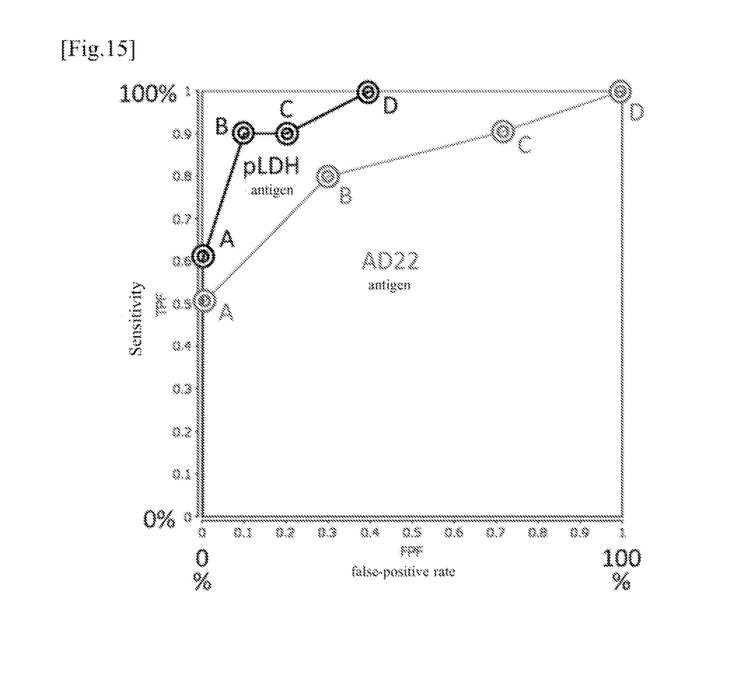

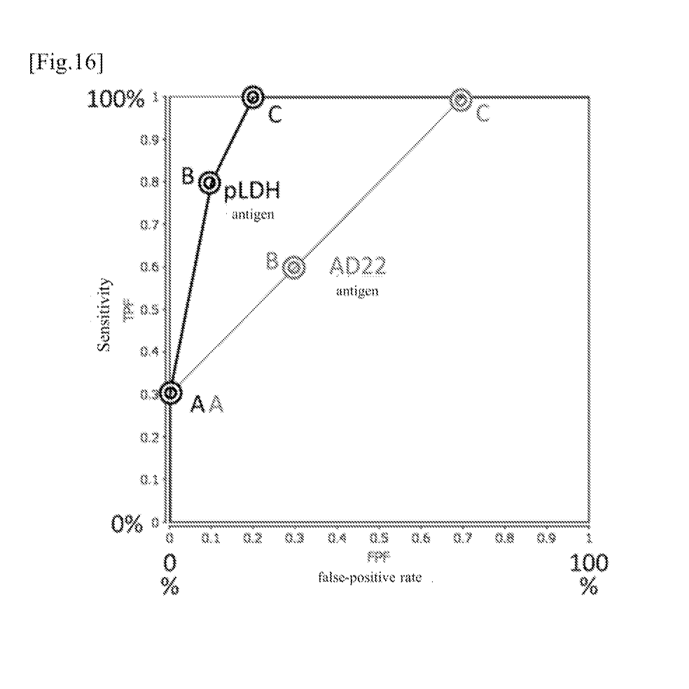

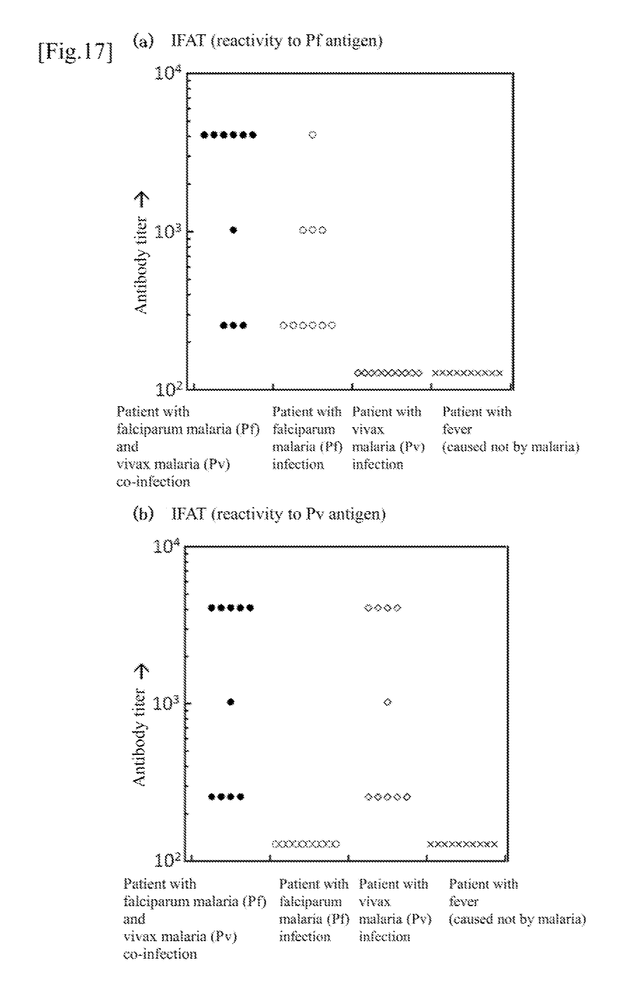

The usability of the inventors' method of the present invention (pLDH microparticle) and the conventional method (a method of a previous invention (AD22 microparticle) by the inventors) as a diagnostic method were compared in terms of the sensitivity and the specificity. Each of the microparticles was used for a test and analyzed, as in the above-described agglutination test. The data in terms of the sensitivity and the specificity, from which the usability as a diagnostic method can be confirmed, has been shown in Tables 3, 4 and 5 and FIGS. 14, 15 and 16. That is, Table 3 and FIG. 14 show the differences in sensitivity and false-positive rate in a case where 10 serum samples from patients with falciparum malaria and vivax malaria co-infection and 10 serum samples from patients with fever, which samples had been collected in endemic areas of the Philippines, were measured and subjected to an ROC analysis. Table 4 and FIG. 15 show the differences in sensitivity and false-positive rate in a case where 10 serum samples from patients with falciparum malaria infection and 10 serum samples from patients with fever, which samples had been collected in endemic areas of the Philippines, were measured and subjected to an ROC analysis. Table 5 and FIG. 16 show the differences in sensitivity and pseudo-positive rate in a case where 10 serum samples from patients with falciparum malaria infection, 10 serum samples from patients with vivax malaria infection and 10 serum samples from patients with fever, which samples had been collected in endemic areas of the Philippines, were measured and subjected to an ROC analysis. Table 3 and FIG. 14 indicate that the method of the present invention is superior in both sensitivity and specificity relative to the conventional method. Table 4 and FIG. 15 indicate that the method of the present invention is superior in both sensitivity and specificity relative to the conventional method. Table 5 and FIG. 16 indicate that the method of the present invention is superior in both sensitivity and specificity relative to the conventional method.

TABLE 3

| | | | | Pseudo- |
|---|---|---|---|---|
| | Cut-off value | Sensitivity | Specificity | positive rate |
| pLDH antigen | | | | |
| (Present invention) | | | | |
| A | 2^{8.5} | 90% | 100% | 0% |
| B | 2^{7.5} | 100% | 90% | 10% |
| AD22 antigen | | | | |
| (Prior art) | | | | |
| A | 2^{8.5} | 70% | 100% | 0% |
| B | 2^{7.5} | 100% | 70% | 30% |

TABLE 4

| | | | | Pseudo- |
|---|---|---|---|---|
| | Cut-off value | Sensitivity | Specificity | positive rate |
| pLDH antigen | | | | |
| (Present invention) | | | | |
| A | 2^{8.5} | 60% | 100% | 0% |
| B | 2^{7.5} | 90% | 90% | 10% |
| C | 2^{6.5} | 90% | 80% | 20% |
| D | 2^{5.5} | 100% | 60% | 40% |
| AD22 antigen | | | | |
| (Prior art) | | | | |
| A | 2^{8.5} | 50% | 100% | 0% |
| B | 2^{7.5} | 80% | 70% | 30% |
| C | 2^{6.5} | 90% | 30% | 70% |
| D | 2^{5.5} | 100% | 0% | 100% |

TABLE 5

| | | | | Pseudo- |
|---|---|---|---|---|
| | Cut-off value | Sensitivity | Specificity | positive rate |
| pLDH antigen | | | | |
| (Present invention) | | | | |
| A | 2^{8.5} | 30% | 100% | 0% |
| B | 2^{7.5} | 80% | 90% | 10% |
| C | 2^{6.5} | 100% | 80% | 20% |
| AD22 antigen | | | | |
| (Prior art) | | | | |
| A | 2^{8.5} | 30% | 100% | 0% |
| B | 2^{7.5} | 60% | 70% | 30% |
| C | 2^{6.5} | 100% | 30% | 70% |

The comparison eventually indicated that the method of the present invention using a single antigen was slightly inferior in specificity relative to the IFAT method using antigens derived from the whole body of protozoa. However, the method of the present invention has a huge advantage in that the method of the present invention can analyze multiple samples concurrently (whereas patient samples are surveyed individually with a fluorescence microscope in the IFAT method) and that the test material is stable even at room temperature since it is a synthetic substance (malaria protozoa antigens used in the IFAT method are required to be stored properly in a refrigerator since they are erythrocyte samples).

Furthermore, the method of the present invention is particularly suitable to identify a serum sample from a subject without a history of malaria infection since the method of the present invention is simple. On the other hand, the IFAT method is convenient to find a case with high titers of antibodies against malaria. In fact, in such an endemic area as the Philippines where the number of malaria patients are decreasing due to the progress of its countermeasure efforts on malaria, many cases with low titers of antibodies against malaria are observed and consequently the method of the present invention is believed to have a huge advantage in practical applications, such as use of the method of the present invention in clinical settings, in countermeasure efforts in endemic areas, in epidemiological studies, and the like.

As described above, the antigen microparticle produced according to the present invention is expected as an alternative method of the IFAT method, in which about one day has been conventionally required to diagnose one sample, to allow considerably more samples to be tested concurrently. Furthermore, the present invention is expected to be used for epidemiological studies on inhabitants in endemic areas and for an investigation on the temporal transmission of an epidemic between populations because 3 μL of a serum sample can produce a result within 5 hours through the present invention.

The IFAT method is a technology to diagnose a current state and recent history of malaria from high titers of antibodies against malaria protozoa. Moreover, an alternative method of the IFAT method is strongly desired in clinical settings because the IFAT method is no longer implemented in Japan, which alternative method exactly diagnoses a fever that one has/had while/after staying in a malaria endemic area and determines whether the fever is/was caused by malaria or not.

The present invention provides a technology to deal with such a demand.

### INDUSTRIAL APPLICABILITY

The novel antigen peptide produced according to the present invention is useful in a field such as medical treatment, diagnosis, research and the like, and can be used particularly in the diagnosis of infection with each of falciparum malaria and vivax malaria, determination of immune state, and determination of presence or absence of an epidemic of malaria (particularly observing the end of an epidemic).

More specifically, the novel antigen peptide can be applied in the following test kit:

a malaria test kit which does not require freezing and refrigerating equipment for transportation and/or storage and any special equipment and/or a power source during measurement and thus can be used even in an endemic area and/or even at bedside;

an infection test kit which is used to diagnose a malaria patient in an endemic area and/or an imported malaria patient in a non-endemic area (which infection test kit is useful as a measure aimed at inhabitants in endemic areas to deal with malaria and useful in inspections directed to overseas travelers returned from endemic areas).

1. An antibody test material for anti-malaria protozoa antibodies, comprising as an active ingredient a peptide comprising the amino acid sequence of SEQ ID NO: 3.

2. The antibody test material according to claim 1, which is for testing anti-falciparum malaria antibodies and an anti-vivax malaria antibodies.

3. The antibody test material according to claim 1, wherein the peptide is immobilized on a carrier.

4. The antibody test material according to claim 3, wherein the carrier is a polymer obtained by a polymerization reaction of compounds (I) and (II) below: 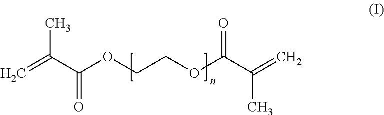
n represents an integer of to 4, 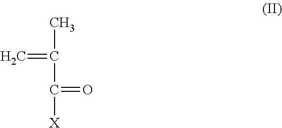
wherein X represents a halogen or —OY; Y represents an alkyl group, aromatic group, pyridyl group, quinolyl group, succinimide group, maleimide group, benzoxazole group, benzothiazole group, or benzotriazole group, wherein a hydrogen atom(s) in these groups may be substituted by a halogen(s).

5. A test agent or diagnostic agent for infection with malaria protozoa, comprising the antibody test material according to claim 1.

6. A test kit or diagnostic kit for infection with malaria protozoa, comprising the antibody test material according to claim 1.

7. A method for testing or diagnosing infection with malaria protozoa, comprising the step of allowing the antibody test material according to claim 1 to react with a sample derived from a subject infected by malaria protozoa or a subject suspected of malaria infection.

8. The method according to claim 7, wherein the antibody test material is for testing anti-falciparum malaria antibodies and anti-vivax malaria antibodies.

9. The method according to claim 7, wherein the peptide is immobilized on a carrier.

10. The method according to claim 9, wherein the carrier is a polymer obtained by a polymerization reaction of compounds (I) and (II) below: 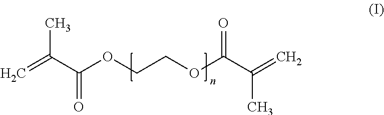
n represents an integer of 1 to 4 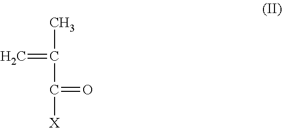
wherein X represents a halogen or —OY; Y represents an alkyl group, aromatic group, pyridyl group, quinolyl group, succinimide group, maleimide group, benzoxazole group, benzothiazole group, or benzotriazole group, wherein a hydrogen atom(s) in these groups may be substituted by a halogen(s).
